(19) United States
(12) Patent Application Publication
(10) Pub. No.: US 2005/0109954 A1
(43) Pub. Date: May 26, 2005

(54) Apparatus and method for verification of outgassing products

(76) Inventors: Waltraud Herbst, Uttenreuth (DE); Karl Kragler, Erlangen (DE); Michael Sebald, Weisendorf (DE)

Correspondence Address: EDELL, SHAPIRO, FINNAN & LYTLE, LLC, 1901 RESEARCH BOULEVARD, SUITE 400, ROCKVILLE, MD, 20850, US

(21) Appl. No.: 10/975,091

(22) Filed: Oct. 28, 2004

(30) Foreign Application Priority Data
Oct. 30, 2003 (DE) DE 103 50 686.1

(51) Int. Cl. A61N 5/00

(52) U.S. Cl. 250/492.1; 250/492.3

(57) ABSTRACT

The outgassing products, which are formed when photoresist systems, are exposed to laser radiation are verified by a mass spectrometer connected to the irradiation chamber.

### CROSS REFERENCE TO RELATED APPLICATIONS

This application claims priority under 35 USC §119 to German Application No. DE 103 50 686. 1, filed on Oct. 30, 2003, and titled “Apparatus and Method for Verification of Outgassing Products,” the entire contents of which are hereby incorporated by reference.

### FIELD OF THE INVENTION

The invention relates to an apparatus and method for verification of outgassing products which are produced by the exposure of photoresists.

### BACKGROUND

Microchips are produced in a large number of process steps, in which changes are deliberately made within a small section of the surface of a substrate, generally a silicon wafer, in order, for example, to introduce trenches for deep-trench capacitors into the substrate, or in order to deposit thin interconnects or electrodes on the substrate surface.

In order to make it possible to display such small structures, a mask is produced on the substrate surface so that those areas, which are intended to be processed, are exposed, while the other areas are protected by the material of the mask. After processing, the mask is removed from the substrate surface again, for example, by incineration.

The mask is produced by applying a thin layer of a photoresist, which has a film-forming polymer as well as a photosensitive compound. This film is subsequently exposed, with a mask, for example, being introduced into the beam path. The mask has the information about the structure to be produced and is used for selective exposure of the photoresist film. For production purposes, the mask is projected onto the photoresist film via a high-resolution lens system.

Photoresist systems are currently subject to rapid technical developments and have major financial importance. The exposure for structuring of photoresists requires complex and expensive beam optics.

Difficulties can occur when radiation at a short wavelength is used for exposing the photoresist. Even at an exposure wavelength of 248 nm, and particularly, at even shorter wavelengths, the high energy of the illumination radiation breaks bonds in the polymer. For example, the photon energy of 7.9 eV at 157 nm is above typical bonding energies of resist polymers, and the photon energy in the EUV band (extreme ultraviolet) with wavelengths around 13 nm is, for example, 95 eV. Polymer systems for exposure wavelengths of 248 nm and below release gaseous decomposition products, which have silicon or other decomposition products, which are damaging to lens systems.

Decomposition products, which have silicon, can then slowly be converted to silicon dioxide by the residual oxygen present in the flushing gas. The silicon dioxide can be precipitated onto the exposure optics and can “blind” the optics over the course of time. Damage and contamination of the lens systems resulting from decomposition products have an adverse effect on the optical characteristics and the quality of the mask structure formed. This contamination may even lead to irreversible damage to the lenses. This results in replacement costs for the damaged optical systems, and in maintenance costs caused by the production failure.

To be able to investigate the behavior of photoresist systems during exposure and the formation of outgassing products is necessary. The corresponding investigation results may then provide opportunities to carry out chemical adaptation to the photoresist or to institute apparatus measures for protection of the lens systems.

Since the rate of development in photoresist technology is high and is increasing further, it is necessary to obtain appropriate information about the outgassing behavior of the photoresist quickly and reliably.

It is possible to irradiate photoresist systems with an electron beam in a vacuum and to gather the outgassing products by a refrigerated trap. The frozen-out materials are then vaporized separately and can be analyzed by mass spectrometry.

However, an incomplete picture of the compounds, which are produced during the exposure process is obtained due to the short life of some compounds or possible subsequently occurring rearrangement or decomposition processes. Furthermore, electron bombardment cannot be transferred to exposure with photons in an unrestricted manner. Also, a large amount of time required for this method.

An apparatus, which allows photoresist systems to be investigated quickly and efficiently with regard to the outgassing products produced during exposure, is desirable.

### SUMMARY

An apparatus for verification of outgassing products can include a radiation source for emitting radiation, a radiation guide for the radiation emitted from the radiation source, an irradiation chamber with a substrate disposed thereon and to which the radiation is applied, and a verification apparatus connected to the radiation source for online verification of the outgassing products emitted from the substrate.

The radiation source, for example, a laser, produces the radiation energy, which is required for exposure. The resolution of the exposure can be varied in a suitable manner by selection of an appropriate wavelength. In particular, wavelengths of 193 nm, 157 nm are currently being used, for example, in microelectronics with 13.4 nm, for instance, to be used in the future.

The radiation guide is used to focus the radiation and to align it on the desired irradiation area. Furthermore, the radiation density and the exposure intensity can be adjusted in a suitable manner by appropriate widening or narrowing of the beam path.

The substrate to be exposed is located, isolated from the rest of the system components, in an irradiation chamber, with the characteristics of the atmosphere within the irradiation chamber, i.e., the pressure, temperature, and gas composition, being chosen appropriately for the requirements.

A verification appliance, which is connected to the radiation source, is connected to the irradiation chamber and detects the compounds, which are released during exposure of the substrate. The connection between the radiation source and the verification appliance allows the verification of the outgassing products to be correlated with the irradiation time periods, and thus the outgassing products to be verified online.

In one embodiment of the apparatus, a dosimeter is provided behind the substrate. The dosimeter measures radiation energy acting on the substrate to record the radiation energy that occurs during the irradiation process, and linked to open-loop and/or closed-loop control processes. The dosimeter may also be installed in the beam guide via beam splitters. Dose monitoring during the analysis process is thus possible.

Individual components of the apparatus can be isolated from one another, in order, for example, to keep the susceptibility to errors as small as possible by a small overall surface area, with relatively precisely set pressures.

In order to ensure an unimpeded radiation profile despite the mutual isolation between the individual components, radiation-transparent windows are provided at the junctions between the components.

The substrate, for example, can be a photoresist.

The photoresist can, for example, be a chemically enhanced photoresist. Chemically enhanced photoresists have photolabile photoacids, which can release disturbing decomposition products when exposed. The acidic products, which are released, can attack and damage the glass materials of the lenses. In the case of photoresist systems, which are subsequently enhanced chemically by organosilicon compounds, silicon salts are formed, in particular, for example, on the lens systems, which are resistive, difficult to remove, and can adversely affect the optical quality of the lenses.

The radiation can, for example, be at a wavelength of less than 200 nm. The resolution of the mask structure to be produced and hence the size of the microelectronic components to be formed are scaled with the wavelength of the radiation used. The integration density of the present-day generation requires a wavelength, which is, for instance, less than 200 nm. In future generations, requirements maybe more stringement.

As the wavelength decreases, the amount of energy transported by the photons rises. The greater this amount of energy, the greater the probability of bonds within the polymers used in the photoresist being broken down. An amount of energy which is greater than the bonding energy of conventional resist polymers occurs even at a wavelength of 157 nm and a photon energy of 7.9 eV. In the EUV (extreme ultra violet) band, which is becoming increasingly important, the photon energy of 95 eV is considerably greater and can thus increasingly cause inadvertent bonding breakdowns in the resist.

In a further embodiment of the invention, the verification apparatus is a mass spectrometer.

Mass spectrometers are physical analysis methods with a very low verification limit and high resolution. Furthermore, the samples to be analyzed can be analyzed very quickly, i.e., in some circumstances even while being exposed. At the same time, he required analysis times are also short and the risk of changes to the substances to be tested during analysis is low.

This method is a physical measurement method, where the charge/mass radio is the characteristic and detected variable. Influencing the samples by chemical pretreatments is thus largely precluded. The molecules to be analyzed are ionized, for example, by an electron beam, and are accelerated to a defined energy in an electrical field. The accelerated and charged particles then enter a magnetic field aligned at right angles to the trajectory, and are deflected by different amounts by the Lorentz force, corresponding to their mass/charge ratio.

A mass selection process then takes place on the particles to be analyzed, with an accuracy of less than one atomic mass unit.

This method makes it possible to obtain a map of the sample composition, at an accurate time, without delay and uncorrupted, at the time of the measurement, and to produce an accurate profile of the compounds released during the exposure.

The mass spectrometer and the irradiation chamber can, for example, have a common vacuum. The compounds, which are formed during exposure, can then be introduced into the mass spectrometer, and can be analyzed, directly and without any delay. In the case of a mass spectrometer, with the vacuum, disturbing the trajectories of the charged sample molecules can be avoided, thus inducing measurement errors. In this case, the common vacuum should be restricted to the area of the irradiation chamber and of the mass spectrometer, in order to keep the sensitive vacuum area as small as possible, and to minimize disturbance sources.

The vacuum can be a hard vacuum or an ultrahard vacuum. The mean free path length of the molecules in these vacuums is relatively long so that collisions between the sample molecules or with other molecules are improbable. The risk of disturbances as a result of secondary reaction or trajectory discrepancies within the mass spectrometer is thus reduced.

The distance between the substrate and an input of the mass spectrometer can, for example, be 5 cm or less. Distances as short as these increase the probability of detection, since the concentration of exposure products in the area close to the substrate is relatively high so that relatively more sample molecules are introduced into the mass spectrometer.

Furthermore, because the path length is short, the probability of collisions with other molecules is low, although it is impossible to preclude a change to the sample composition as a result of possible secondary reactions. At the same time, with the short dwell time in the irradiation chamber, an accurate image of the local composition of the exposure products can be obtained, since unimolecular rearrangements over very short time intervals are of relatively minor importance.

The radiation guide, the substrate, and the input of the mass spectrometer can for example, form an angle of 30° to 60°. The probability for verification in the mass spectrometer is increased in this angle range, since resist fragments generally outgas at right angles to the resist.

In a further embodiment, the substrate is introduced into the irradiation chamber via a sample inlet.

The apparatus according to the invention can be used in a method for verification of irradiation products. Carrying out such a method online provides an opportunity to follow the time concentration profile of the irradiation products directly, and thus to draw conclusions relating to the causes of the creation of the outgassing compounds formed. Furthermore, suitable parameters, such as the exposure intensity, duration, temperature, pressure, etc., can be changed during the measurement, and their direct effect on the emission of outgassing products can be studied.

### BRIEF DESCRIPTION OF THE FIGURES

The invention will be explained in more detail using an exemplary embodiment and with reference to the attached figures, in which, in detail:

FIG. 1 shows a schematic configuration of the apparatus according to the invention, and

FIG. 2 shows an embodiment of the apparatus according to the invention, in use.

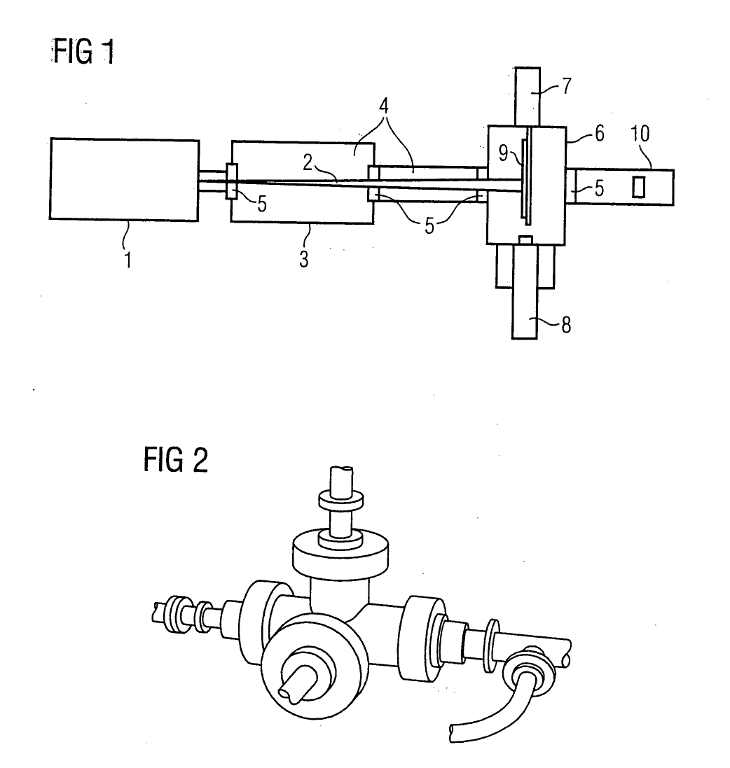

### DETAILED DESCRIPTION

A laser 1 emits a laser beam 2 at a wavelength of, for example, 193 nm, which is suitable for exposure. The laser beam 2 passes through a radiation guide 4, which may include one or more laser-optical chambers 3. The laser beam 2 is focused in accordance with the requirements, and/or is widened and aligned with the area to be irradiated, in this radiation guide 4.

The area to be irradiated is located on a photoresist 9. The photoresist 9 is introduced into the irradiation chamber 6 via a sample inlet 7.

The input to the mass spectrometer 8, which is arranged on the side of the irradiation chamber 6, is located in the immediate vicinity of the photoresist 9.

A dosimeter 10 for measurement of the radiation dose is located on an imaginary linear continuation of the laser beam 2 behind the irradiation chamber 6.

The radiation guide 4, the irradiation chamber 6 with the mass spectrometer 8 as well as the dosimeter 10 in this case form vacuum systems which are isolated from one another. In order to ensure that the radiation can pass through, windows 5 are provided between the individual components, through which the wavelength of the laser light at, for example, 193 nm, can pass.

The laser beam 2 is emitted from the laser 1, is focused by the radiation guide 4, and strikes the photoresist 9. Exposure products are formed on the surface of the photoresist 9, which outgas into the vacuum within the irradiation chamber 6.

These outgassing products enter the inlet opening of the mass spectrometer 8, and are then analyzed. A continuous concentration profile of the outgassing products can be recorded throughout the course of the exposure of the photoresist.

The dosimeter 10 monitors whether the exposure procedure is being carried out correctly, with regard to the magnitude and homogeneity of the exposure energy. Therefore, fluctuations, which may occur in the measured concentration profiles of the outgassing products can be accounted for.

While the invention has been described in detail and with reference to specific embodiments thereof, it will be apparent to one skilled in the art that various changes and modifications can be made therein without departing from the spirit and scope thereof. Accordingly, it is intended that the present invention covers the modifications and variations of this invention provided they come within the scope of the appended claims and their equivalents.

### List of Reference Symbols

1 Radiation source
2 Radiation
3 Laser-optical chamber
4 Radiation guide
5 Window
6 Irradiation chamber
7 Sample inlet
8 Verification apparatus
9 Substrate
10 Dosimeter

1. An apparatus for verification of outgassing products, comprising:
a radiation source for emitting radiation;
a radiation guide for the emitted radiation;
an irradiation chamber with a substrate, the substrate being arranged thereon, radiation being applied to the substrate; and
a verification apparatus connected to the radiation source for online verification of the outgassing products emitted from the substrate.

2. The apparatus as claimed in claim 1, further comprising:
a dosimeter for measuring the radiation.

3. The apparatus as claimed in claim 1, further comprising:
at least one radiation-transparent window arranged between components.

4. The apparatus as claimed in claim 1, wherein the substrate is a photoresist.

5. The apparatus as claimed in claim 4, wherein the photoresist is a chemically enhanced photoresist.

6. The apparatus as claimed in claim 1, wherein the radiation is at a wavelength of less than 200 nm.

7. The apparatus as claimed in claim 1, wherein the verification apparatus is a mass spectrometer.

8. The apparatus as claimed in claim 7, wherein the mass spectrometer and the irradiation chamber have a common vacuum.

9. The apparatus as claimed in claim 8, wherein the vacuum is a hard vacuum or an ultrahard vacuum.

10. The apparatus as claimed in claim 2, further comprising:
a distance of 5 cm or less between the substrate and an input of a mass spectrometer.

11. The apparatus as claimed in claim 1, wherein the radiation guide, the substrate, and an input of a mass spectrometer form an angle ranging between 30° and 60°.

12. The apparatus as claimed in claim 1, wherein the substrate is introduced into the irradiation chamber via a sample inlet.

13. A method for verification of irradiation products, comprising:
using an apparatus for verification of outgassing products, the apparatus including
a radiation source for emitting radiation,
a radiation guide for the emitted radiation,
an irradiation chamber with a substrate, the substrate being arranged therein, radiation being applied to the substrate; and
a verification apparatus connected to the radiation source for online verification of the outgassing products emitted from the substrate.
